(19) United States
(12) Patent Application Publication
(10) Pub. No.: US 2025/0248731 A1
(43) Pub. Date: Aug. 7, 2025

(54) BLADE CHAMFER AND/OR TAPER ON CUTTING TIP FOR IMPLANTED LEAD REMOVAL WITH MINIMIZED TISSUE COMPRESSION

(71) Applicant: KONINKLIJKE PHILIPS N.V., EINDHOVEN (NL)

(72) Inventor: CLINT BRUBAKER, COLORADO SPRINGS, CO (US)

(21) Appl. No.: 19/039,936

(22) Filed: Jan. 29, 2025

Related U.S. Application Data
(60) Provisional application No. 63/549,696, filed on Feb. 5, 2024.

(51) Int. Cl. A61B 17/32

(52) U.S. Cl. CPC A61B 17/32002

(57) ABSTRACT

A lead removal device includes a flexible elongate member configured to be positioned within a blood vessel of a patient, and a cutting tip positioned at the distal portion of the flexible elongate member and configured to rotate. The cutting tip includes: a blade configured to cut tissue associated with the blood vessel for removal of an electrical lead implanted in the tissue from a body of a patient, an outer wall surface defining an outer diameter, and an inner wall surface defining a lumen with an inner diameter, wherein the lumen is configured to receive the electrical lead and the tissue cut by the blade. The cutting tip has an outward chamfer such that a cutting edge of the blade is aligned with the inner wall surface, and the inner diameter matches the diameter of the tissue cut by the blade and entering the lumen.

### CROSS REFERENCE TO RELATED APPLICATION

This application claims the benefit of U.S. Provisional Application No. 63/549,696 filed Feb. 5, 2024. This application is incorporated by reference herein.

### TECHNICAL FIELD

The subject matter described herein relates to a cutting tip of a cutting catheter (e.g., a lead removal device). The cutting tip is sized and shaped to prevent jamming of the cutting tip when tissue that is cut by the cutting tip enters the cutting tip. This apparatus has particular but not exclusive utility for removal of implanted leads from a body lumen of a patient.

### BACKGROUND

Surgically implanted cardiac pacing systems, such as pacemakers and defibrillators, play an important role in the treatment of heart disease. Pacemakers treat slow heart rhythms by increasing the heart rate or by coordinating the heart's contraction for some heart failure patients. Implantable cardioverter-defibrillators stop dangerous rapid heart rhythms by delivering an electric shock. Cardiac pacing systems typically include a timing device and a lead, which are placed inside the body of a patient. One part of the system is the pulse generator containing electric circuits and a battery, usually placed under the skin on the chest wall beneath the collarbone. Another part of the system includes the wires, or leads, which run between the pulse generator and the heart.

These leads must be in contact with heart tissue. To remain attached to the heart muscle, most leads have a fixation mechanism, such as a small screw and/or hooks at the end. Within a relatively short time after a lead is implanted into the body, the body's natural healing process forms scar tissue along the lead and possibly at its tip, thereby fastening it even more securely in the patient's body. Leads usually last longer than device batteries, so leads are simply reconnected to each new pulse generator (battery) at the time of replacement. Although leads are designed to be implanted permanently in the body, occasionally these leads must be removed, or extracted. Leads may be removed from patients for numerous reasons, including but not limited to, infections, lead age, and lead malfunction.

Removal or extraction of the lead may be difficult. As mentioned above, the body's natural healing process forms scar tissue over and along the lead, and possibly at its tip, thereby encasing at least a portion of the lead and fastening it even more securely in the patient's body. In addition, the lead and/or tissue may become attached to the vasculature wall. Both results may, therefore, increase the difficulty of removing the leads from the patient's vasculature.

A variety of tools have been developed to make lead extraction safer and more successful. A mechanical device to extract leads may include one or more flexible tubes called sheaths that passes over the lead and/or the surrounding tissue. One of the sheaths may include a tip having a dilator, a separator and/or a cutting blade, such that upon advancement, the tip cuts or dilates the scar tissue to separate the scar tissue from other scar tissue, including the scar tissue surrounding the lead. In some cases, the tip (and sheath) may also separate the tissue itself from the lead. Once the lead is separated from the surrounding tissue and/or the surrounding tissue is separated from the remaining scar tissue, the lead may be inserted into a hollow lumen of the sheath for removal and/or be removed from the patient's vasculature using some other mechanical devices, such as mechanical traction devices.

Some current lead removal devices include of a handle that transmits torque and rotation to the proximal end of a long shaft. In some versions, the device is used by squeezing the trigger, resulting in an extension and rotation of cutting blades. In other versions, the extension and rotation of the blades is controlled by a motor and electronics in the handle. The cutting blades remove body tissue that would otherwise prevent the extraction of pacemaker leads. The blades of these devices were designed to cut through the lesion material and allow the lead to be extracted. The design of the blades has a ring of cutting teeth around the outer diameter that then tapers down to the inner diameter of the cutter.

However, one of the main issues encountered in using such lead removal devices is stuck or jammed leads. A stuck or jammed lead occurs during a procedure when the device cuts through a lesion adhered to a lead. After cutting partway or through the lesion, the lesion and lead get stuck within the device, leading to the inability to pull out the lead from the device and/or preventing further cutting of the lesion.

The information included in this Background section of the specification, including any references cited herein and any description or discussion thereof, is included for technical reference purposes only and is not to be regarded as subject matter by which the scope of the disclosure is to be bound.

### SUMMARY

The present disclosure provides an intraluminal cutting device with several advantageous features. One such feature is an outward-chamfered cutting tip that tends to push cut tissue radially outward rather than compressing it inward. Another such feature is an offset blade cutting tip or dual-chamfered cutting tip, which includes an outer row of cutting teeth aligned with the outer diameter of the cutting tip and an inner row of cutting teeth aligned with the inner diameter of the cutting tip, such that the cut size of a tissue plug is larger than the diameter of the plug, thus limiting compression of tissue as it is pushed into the cutting catheter tip. Still another advantageous feature of the present disclosure is a cutting tip that includes an inside taper, wherein the inner diameter of the cutting tip increases in the proximal direction, thus allowing any compressed tissue to re-expand as it moves proximally through the cutting tip. All three of these aspects reduce the amount of compression the tissue experiences as the cutting tip is advanced over the implanted lead, and thus reduce the amount of compression-related friction on the intraluminal cutting device, limiting the likelihood that the cutting device will jam.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a lead removal device. The lead removal device includes a flexible elongate member configured to be positioned within a blood vessel of a patient, where the flexible elongate member may include a proximal portion and a distal portion; a cutting tip positioned at the distal portion of the flexible elongate member and configured to rotate, where the cutting tip may include: a blade configured to cut tissue associated with the blood vessel for removal of an electrical lead implanted in the tissue from a body of a patient; an outer wall surface defining an outer diameter; an inner wall surface defining a lumen with an inner diameter, where the lumen is configured to receive the electrical lead and the tissue cut by the blade; and an outward chamfer such that: a cutting edge of the blade is aligned with the inner wall surface; and the inner diameter matches a diameter of the tissue cut by the blade and entering the lumen.

Implementations may include one or more of the following features. In some aspects, the blade may include a plurality of teeth and a plurality of serrations, where a distal end of each tooth may include a cutting edge. In some aspects, each tooth of the plurality of teeth may include a chamfered trapezoidal surface, where each serration of the plurality of serrations may include a chamfered curved surface. In some aspects, the intraluminal cutting device may include: a handle coupled to the proximal portion of the flexible elongate member; and a trigger coupled to the handle and configured such that actuation of the trigger causes the cutting tip to rotate. Implementations of the described techniques may include hardware, a method or process, or computer software on a computer-accessible medium.

One general aspect includes a lead removal device. The lead removal device includes a flexible elongate member configured to be positioned within a blood vessel of a patient, where the flexible elongate member may include a proximal portion and a distal portion; a cutting tip positioned at the distal portion of the flexible elongate member and configured to rotate, where the cutting tip may include: a blade configured to cut tissue associated with the blood vessel for removal of an electrical lead implanted in the tissue from a body of a patient; an outer wall surface defining an outer diameter; an inner wall surface defining a lumen with an inner diameter, where the lumen is configured to receive the electrical lead and the tissue cut by the blade; and an outward chamfer and an inner chamfer such that: a first cutting edge of the blade is aligned with the inner wall surface; a second cutting edge of the rotating blade is aligned with the outer wall surface; and the inner diameter matches a diameter of the tissue cut by the blade and entering the lumen.

Implementations may include one or more of the following features. In some aspects, the blade may include a plurality of teeth and a plurality of serrations, where a distal end of each tooth may include a cutting edge. In some aspects, each tooth of the plurality of teeth may include a chamfered trapezoidal surface, where each serration of the plurality of serrations may include a chamfered curved surface. In some aspects, the intraluminal cutting device may include: a handle coupled to the proximal portion of the flexible elongate member; and, a trigger coupled to the handle and configured such that actuation of the trigger causes the cutting tip to rotate. Implementations of the described techniques may include hardware, a method or process, or computer software on a computer-accessible medium.

One general aspect includes an intraluminal cutting device. The intraluminal cutting device includes a flexible elongate member configured to be positioned within a blood vessel of a patient, where the flexible elongate member may include a proximal portion and a distal portion; a cutting tip positioned at the distal portion of the flexible elongate member and configured to rotate, where the cutting tip may include: a blade configured to cut tissue associated with the blood vessel for removal of an electrical lead implanted in the tissue from a body of a patient; an outer wall surface defining an outer diameter; an inner wall surface defining a lumen with an inner diameter, where the lumen is configured to receive the electrical lead and the tissue cut by the blade, and where the inner wall surface may include a taper such that: the inner diameter at a distal end of the lumen is less than the inner diameter at a proximal end of the lumen; the inner diameter at the proximal end of the lumen is greater than a diameter of the tissue cut by the blade and entering the lumen.

Implementations may include one or more of the following features. In some aspects, the blade may include a plurality of teeth and a plurality of serrations, where a distal end of each tooth may include a cutting edge. In some aspects, each tooth of the plurality of teeth may include a chamfered trapezoidal surface, where each serration of the plurality of serrations may include a chamfered curved surface. In some aspects, the intraluminal cutting device may include: a handle coupled to the proximal portion of the flexible elongate member; and, a trigger coupled to the handle and configured such that actuation of the trigger causes the cutting tip to rotate. In some aspects, the blade is configured such that a cutting edge of the rotating blade is aligned with the outer wall surface. Implementations of the described techniques may include hardware, a method or process, or computer software on a computer-accessible medium.

One general aspect includes an intraluminal cutting device. The intraluminal cutting device includes a flexible elongate member configured to be positioned within a body lumen of a patient, where the flexible elongate member may include a proximal portion and a distal portion; a cutting tip positioned at the distal portion of the flexible elongate member and configured to rotate, where the cutting tip may include: a blade configured to cut tissue associated with the body lumen, an outer wall surface defining an outer diameter; an inner wall surface defining a lumen with an inner diameter. The cutting tip may include a structural arrangement configured to minimize compression of the tissue cut by the blade and entering the lumen, where the minimization of the compression may include at least one of minimization of compression-related friction of the blade or prevention of jamming of the blade. The structural arrangement may include at least one of: the cutting tip may include an outward chamfer or both the outward chamfer and an inward chamfer, such that the inner diameter matches a diameter of the tissue cut by the blade and entering the lumen; or the inner wall surface may include a taper such that: the inner diameter at a distal end of the lumen is less than the inner diameter at a proximal end of the lumen; and the inner diameter at the proximal end of the lumen is greater than a diameter of the tissue cut by the blade and entering the lumen.

Implementations may include one or more of the following features. In some aspects, the blade may include a plurality of teeth and a plurality of serrations, where a distal end of each tooth may include a cutting edge. In some aspects, each tooth of the plurality of teeth may include a chamfered trapezoidal surface, where each serration of the plurality of serrations may include a chamfered curved surface. In some aspects, the intraluminal cutting device may include: a handle coupled to the proximal portion of the flexible elongate member; and, a trigger coupled to the handle and configured such that actuation of the trigger causes the cutting tip to rotate. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the intraluminal cutting device, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic schematic view of an intraluminal cutting device or surgical device, such as a cutting catheter device, according to aspects of the present disclosure.

FIG. 2 is a side perspective view of an example surgical device, according to aspects of the present disclosure.

FIG. 3 is a side cross-sectional view of a distal portion of an example sheath assembly, according to aspects of the present disclosure.

FIG. 4 is a perspective side view of the distal end of an example sheath assembly, according to aspects of the present disclosure.

FIG. 5 is a perspective side view of the distal end of an example sheath assembly, according to aspects of the present disclosure.

FIG. 6 is a diagrammatic view of a surgical device that has been introduced into a body lumen of a patient to remove an implanted lead, according to aspects of the present disclosure.

FIG. 7 is a side cross-sectional view of an example sheath assembly removing a lead from a body lumen, according to aspects of the present disclosure.

FIG. 8 is a side front perspective view of an example cutting tip, according to aspects of the present disclosure.

FIG. 9 is a schematic cross-sectional view of the reciprocating motion of a cutting tip of the distal portion of an example sheath assembly, according to aspects of the present disclosure.

FIG. 10 is a schematic cross-sectional view of the reciprocating motion of a cutting tip of the distal portion of the example sheath assembly of FIG. 9, according to aspects of the present disclosure.

FIG. 11 is a schematic cross-sectional view of the reciprocating motion of a cutting tip of the distal portion of the example sheath assembly of FIG. 10, according to aspects of the present disclosure.

FIG. 12 is a side cross-sectional view of the cutting tip of FIG. 8, according to aspects of the present disclosure.

FIG. 13 is a schematic, diagrammatic, side cross-sectional view of an example cutting tip cutting into tissue surrounding a lead, according to aspects of the present disclosure.

FIG. 14 is a graph showing a curve of compressive friction force as a function of the cut depth of the cutting tip into the tissue surrounding the lead, according to aspects of the present disclosure.

FIG. 15 is a side perspective view of an example cutting tip that incorporates an outward chamfer or positive chamfer, according to aspects of the present disclosure.

FIG. 16 is a side cross-sectional view of an example cutting tip that incorporates an outward chamfer or positive chamfer, according to aspects of the present disclosure.

FIG. 17 is a schematic, diagrammatic, side cross-sectional view of an example outward-chamfered cutting tip cutting into tissue surrounding a lead, according to aspects of the present disclosure.

FIG. 18 is a graph showing a curve of compressive friction force as a function of the cut depth of the outward-chamfered cutting tip into the tissue surrounding the lead, according to aspects of the present disclosure.

FIG. 19 is a side perspective view of an example cutting tip that incorporates a dual chamfer or positive-negative chamfer, according to aspects of the present disclosure.

FIG. 20 is a side cross-sectional view of the dual-chamfered cutting tip of FIG. 19, according to aspects of the present disclosure.

FIG. 21 is a schematic, diagrammatic, side cross-sectional view of an example dual-chamfered cutting tip cutting into tissue surrounding a lead, according to aspects of the present disclosure.

FIG. 22 is a graph showing a curve of compressive friction force as a function of the cut depth of the dual-chamfered cutting tip into the tissue surrounding the lead, according to aspects of the present disclosure.

FIG. 23 is a side cross-sectional view of an example cutting tip with an inward or negative chamfer and a tapered interior lumen, according to aspects of the present disclosure.

FIG. 24 is a schematic, diagrammatic, side cross-sectional view of an example inward-chamfered cutting tip with tapered lumen cutting into tissue surrounding a lead, according to aspects of the present disclosure.

FIG. 25 is a graph showing a curve of compressive friction force as a function of the cut depth of the dual-chamfered cutting tip into the tissue surrounding the lead, according to aspects of the present disclosure.

FIG. 26 is a schematic diagram of a processor circuit, according to aspects of the present disclosure.

### DETAILED DESCRIPTION

Currently, mechanical products are used in lead management/removal procedures. These devices may include a handle that transmits torque and rotation to the proximal end of a cutting catheter, whose distal end includes a cylindrical cutting tip with a sharp or serrated lip forming one or more blades. In some aspects, the device is actuated by applying a squeezing force to the trigger, resulting in an extension and rotation of the cutting blades. In other aspects, the extension and rotation of the blades is controlled by a motor and electronics in the handle of the device. The cutting blades remove body tissue that would otherwise prevent the extraction of pacemaker leads. The blades of these devices are designed to cut through the lesion material and allow the lead to be extracted. The design of the blades can include a ring of cutting teeth around the outer diameter of the cutting tip that then tapers down (e.g., at a 60-degree angle) to the inner diameter of the cutting tip.

One issue encountered in using such cutting tips is sticking or jamming of electrical leads within the cutting tip. A stuck or jammed lead occurs during a procedure when the device cuts through a lesion adhered to a lead. After the tip has cut partway through the lesion, the lesion and lead get stuck within the tip, leading to an inability to pull out the lead from the device and/or preventing further cutting of the lesion.

One cause of this problem is with the current blade profile. Because of the inward chamfer of the tip, the outer diameter of the plug of material that is cut and pulled into the lumen of the cutter/shaft is larger than the inner diameter of the cylindrical cutting tip. In one non-limiting example, on some devices that have encountered jamming issues, the cut plug reduces from the cut diameter of 0.205 inches to 0.171 inches in diameter of the inner cutting tip lumen, which equates to a compression of 16.6%. In some devices, the compression is constant through the shaft due to the straight shaft design, where the inner diameter is consistent throughout the entire device. This leads to an effect where the further the device cuts into the lesion material, the more frictional resistance it encounters. However, in other exemplary devices, the plug only has to reduce from the cut diameter of 0.191 to 0.171 inches, thus compressing 10.5%.

In addition, in some devices, proximal of the cutting tip, the catheter shaft opens up to a diameter of 0.183 inches, bringing the plug compression down to 4.2%. It has been observed that such devices can cut much farther in clinical use and testing than devices with a greater degree of compression. Accordingly, a need exists to reduce or eliminate this compressive action during cutting on mechanical lead removal devices, to improve the performance of devices and to reduce clinical complaints. The present disclosure provides an intraluminal cutting device with several advantageous features.

One such feature is an offset blade cutting tip. On many saws used to cut material, one of the main features of the blade design is an offset pattern to the teeth. On a bandsaw blade, for example, depending on the pattern, the teeth are bent/shaped so that they alternate on each side of the line of symmetry. Such blades are designed this way so that the cut size made by the teeth is larger than the thickness of the band, which helps prevent the band from getting jammed or stuck when cutting a material.

A similar feature can be incorporated into the cutting tip, as described below. In the pattern, each tooth or blade in the cutting tip can be alternated from being placed on the cutter's outer diameter to the cutter's inner diameter. This design helps ensure that the diameter of the plug is equal to or smaller than the inside diameter of the cutting tip, thus reducing or eliminating friction caused by compression of the tissue plug. The blades can be straight, or can be bent inwards to further reduce the plug's diameter. To build the offset blade cutter, existing cutting tip designs can be modified for machining to have alternating offset blades. The new cutter design can then be incorporated into existing or novel mechanical lead removal devices.

Another advantageous feature of the present disclosure is a cutting tip that incorporates an outside chamfer. Many current cutting tip designs include a chamfer from the blade teeth inwards to the inner diameter. The outside chamfer of the present disclosure reverses this narrowing, both to push out the outer material and to leave the plug the same diameter as the inner diameter of the shaft, thus eliminating or dramatically reducing friction caused by compression of the tissue plug. To build the Outside Chamfer cutter, existing cutting tip designs can be modified by machining the chamfer on the outside instead of the inside. The new cutter design can then be incorporated into existing or novel mechanical lead removal devices.

Still another advantageous feature of the present disclosure is a cutting tip that includes an inside taper, wherein the inner diameter of the cutting tip increases in the proximal direction, thus allowing any compressed tissue to re-expand as it moves proximally through the cutting tip and into the catheter itself. To build the tapered cutter, the existing cutting tip designs can be modified by machining the taper on the inside of the cutter. The new cutter design can then be incorporated into existing or novel mechanical lead removal devices.

The present disclosure substantially aids a clinician in removing an implanted lead from a body lumen of a patient, by providing a cutting tip that reduces or eliminates friction caused by compression of the tissue plug as it advances through the cutting tip. The system may include cutting tips with specialized shapes. Implemented on a cutting catheter in mechanical communication with a handle assembly, the intraluminal cutting device disclosed herein may provide both time savings and an improvement in the safety and precision of lead removal procedures. This improved lead removal workflow transforms a slow, painstaking process into one where the risk of friction-related jamming is greatly reduced, without the normally routine need to halt the procedure and remove the cutting catheter. This unconventional approach improves the functioning of the lead removal system, by allowing a single cutting catheter to cut much farther into scar tissue without jamming.

The devices, systems, and methods described herein can include one or more features described in U.S. Pat. No. 10,314,615 and titled “Medical device for removing an implanted object”, U.S. Pat. No. 9,980,743 and titled “Medical device for removing an implanted object using laser cut hypotubes”, U.S. Pat. No. 10,136,913 and titled “Multiple configuration surgical cutting device”, and U.S. Pat. No. 10,993,741 and titled “Surgical cutting device with shield drive mechanism”, each of which is hereby incorporated by reference in their entirety as though fully set forth herein.

Portions of the system disclosed herein may be implemented as a set of logical branches and mathematical operations, whose outputs are viewable on a display, and operated by a control process executing on a processor that accepts user inputs from a trigger, touchscreen interface, or other user interface, and that is in communication with one or more motors controlling rotation of the cutting tip. In that regard, the control process performs certain specific operations in response to different inputs or selections made by a user at different times. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the disclosure. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. For the sake of brevity, however, the numerous iterations of these combinations will not be described separately.

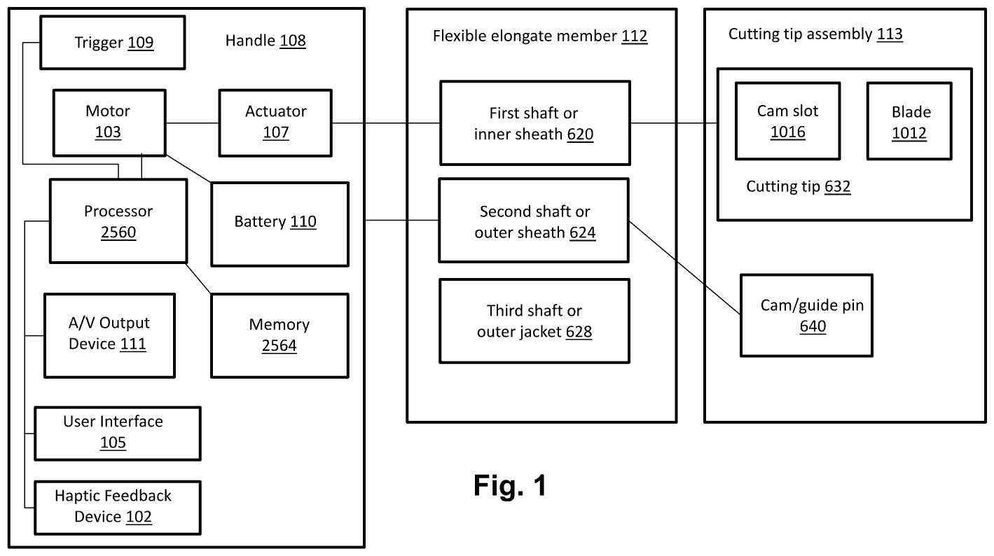

FIG. 1 is a diagrammatic schematic view of an intraluminal cutting device 106 or surgical device 106, such as a cutting catheter device, according to aspects of the present disclosure. The intraluminal cutting device or surgical device 106 includes a handle 108, a flexible elongate member or sheath assembly 112, and a cutting tip assembly 113.

The handle 108 may include a trigger 109, one or more motors 103, an actuator 107, a processor 2560, a battery 110, an audiovisual (A/V) output device 111 (e.g., a display screen, a set of indicator lights, etc.), and a user interface 105 (e.g., a touchscreen, one or more buttons, switches, dials, etc.).

The flexible elongate member or sheath assembly 112 may include a rotatable first shaft or flexible inner sheath 620 coupled to the actuator 107, a fixed second shaft or flexible outer sheath 624 that surrounds the flexible inner sheath 620, and a translatable third shaft or outer jacket 628 that surrounds the flexible outer sheath 624.

In an example, the actuator 107 be or include a power train assembly with one or more gears coupling the 103 motor to the first (inner) shaft or flexible inner sheath 620. A press of the trigger 109 may send a signal to the processor 2560, which then activates the motor 103, which is powered by the battery 110 and transmits rotational motion through the actuator 107 to the flexible inner sheath 620, which then rotates along with the actuator. The flexible outer sheath 624 may be fixed to the handle 108, such that it remains fixed while the flexible inner sheath 620 rotates within it.

The cutting tip assembly 113 may for example include a cutting tip 632 with a cam slot 1016 and blade 1012. The cutting tip 632 is fixedly attached to the flexible inner shaft 620, such that rotation of the flexible inner shaft 620 causes rotation of the cutting tip 632. A cam pin, guide pin, or cam guide pin 640 is fixedly attached to the flexible outer sheath 624, but fits within the cam slot 1016, such that rotation of the cutting tip 632 can also drive axial or longitudinal motion of the cutting tip, as described below.

The third shaft or outer jacket 628 surrounds the second shaft or outer sheath 624, and is translatably movable along it, such that a distal portion of the outer jacket 628 can be advanced to cover the blade 1012 of the cutting tip 632, as described below.

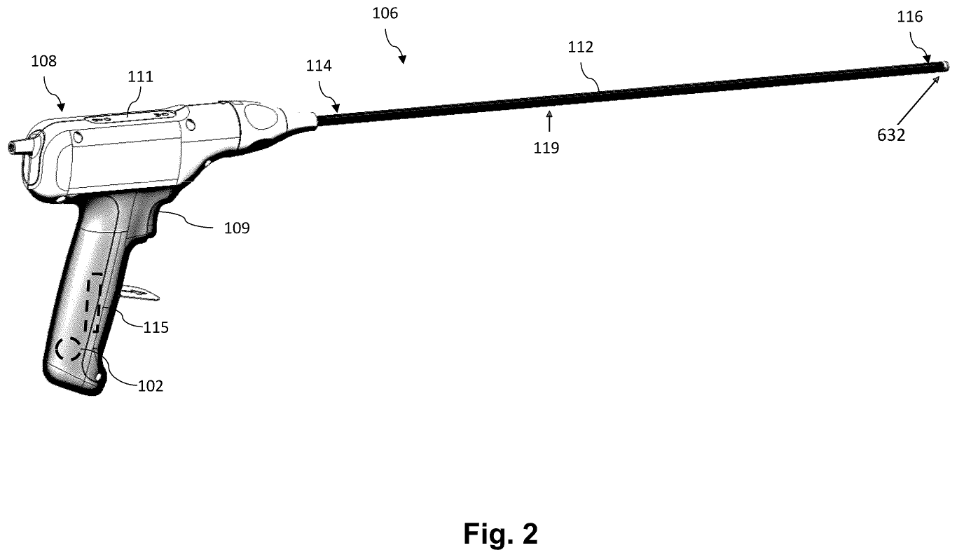

FIG. 2 is a side perspective view of an example surgical device 106, according to aspects of the present disclosure. The surgical device 106 includes a sheath assembly 112 that can be inserted into a body lumen 334 of a patient 104 (see FIG. 6). The sheath assembly includes a proximal portion 114 and a distal portion 118, separated by a working length 119 that is sufficient to perform the tasks described herein. The distal portion 118 includes a movable cutting tip 632.

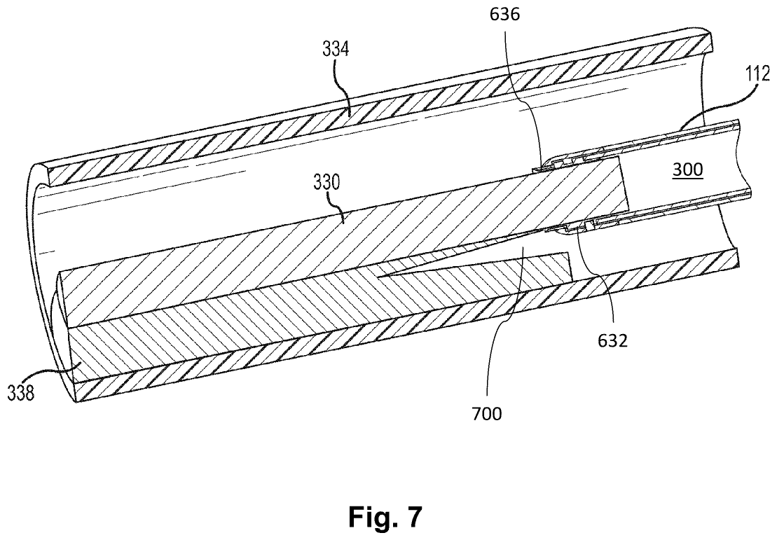

The sheath assembly 112 can surround an implanted lead 330 (see FIG. 7), such as a lead running along the left innominate vein past the superior vena cava (SVC) and connected into, or about, the right ventricle of the heart. Upon surrounding the lead 330 with the sheath assembly 112, the user of the surgical device 106 may actuate the handle assembly 108 (e.g., with a trigger 109), thereby actuating the cutting tip 632 located at the distal end of the sheath assembly 112, as described below. The actuated cutting tip 632, can then separate and/or cut the tissue surrounding an implanted lead within the body lumen of the patient.

Depending on the implementation, the handle assembly 108 may also include audiovisual (A/V) feedback indicators 111, a controller printed circuit board assembly (PCBA) 115, and a haptic feedback device 102.

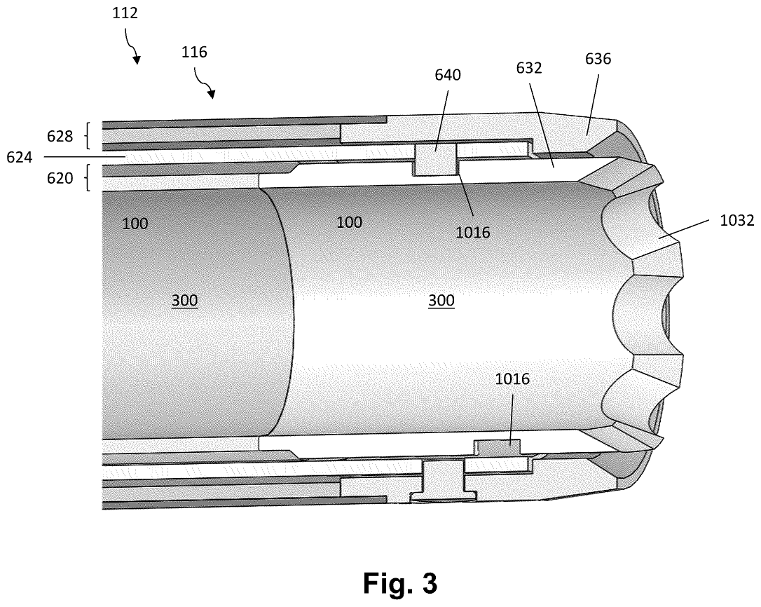

FIG. 3 is a side cross-sectional view of a distal portion 116 of an example sheath assembly 112, according to aspects of the present disclosure. The distal portion 116 of the sheath assembly 112 includes an outer band 636 fixedly attached to a flexible outer jacket 628, and a cutting tip 632 fixedly attached to a flexible inner sheath 620. In the example shown in FIG. 3, the flexible outer sheath 624 surrounds the flexible inner sheath 620, and the outer band 636 surrounds the cutting tip 632. The cutting tip 632 and flexible inner sheath 620 together define an inner lumen 300. A guide pin 640 is fixedly attached to the outer band 636. The cutting tip 632 is rotatably attached to the outer band 636 via the guide pin 640 that rests in a channel or can slot 1016. Activation of the trigger 109 of the handle assembly 108 (see FIG. 2) causes the flexible inner sheath 620 to rotate, whereas the flexible outer sheath 624 is rotationally fixed. The channel or cam slot 1016 is formed in the cutting tip 632 in a profile that varies in longitudinal distance with different radial positions, such that when the flexible inner sheath 620 is rotated while the flexible outer sheath 624 is rotationally fixed, the guide pin 640 travels through the cam slot 1016, causing the flexible inner shaft 620 and the cutting tip 632 to translate longitudinally as they rotate, as will be shown in greater detail below.

Depending on the profile of the cam slot 1016, a serrated blade or cutting surface 1012 of the cutting tip 632 may thus extend from and retract into the outer band 636 multiple times upon actuation of the trigger of the handle assembly. Depending on the implementation, the blade 1012 may rotate in a clockwise direction, a counterclockwise direction, or may oscillate between the two. When the clinician releases the trigger of the handle assembly, the blade 1012 of the cutting tip 632 may retract within the outer band 636, thereby allowing the clinician to force and advance the distal portion of the sheath assembly against additional uncut tissue, without engagement of the tissue by the blade 1012 of the cutting tip 632. The clinician may repeat the actuation step, thereby causing the blade 1012 of the cutting tip 632 to extend distally beyond the outer band 636 to cut the adjacent tissue. Each time actuation occurs, the proximal portion of the implanted lead and/or surrounding tissue enters further into the central lumen 300 of the sheath assembly 112. This process can be repeated until the surrounding tissue is completely or substantially dilated, and the implanted lead is separated and/or cut from the tissue. At that time, the implanted lead may safely be removed from the patient.

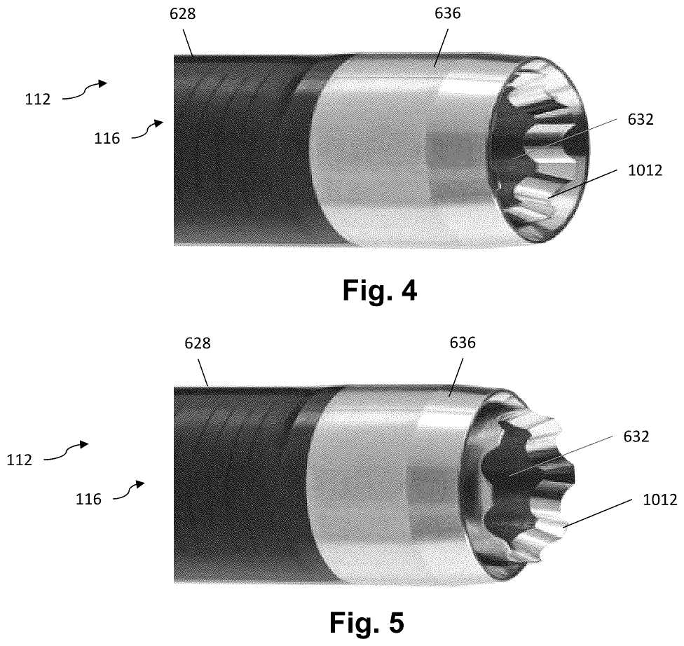

FIG. 4 is a perspective side view of the distal end of an example sheath assembly 112, according to aspects of the present disclosure. In this example, the blade 1012 of the cutting tip 632 is retracted inside the outer band 636. Also visible is the flexible outer jacket 628. In this configuration, the outer band 636 can be advanced against tissue in order to dilate or separate it without cutting (e.g., to separate the tissue from an implanted lead).

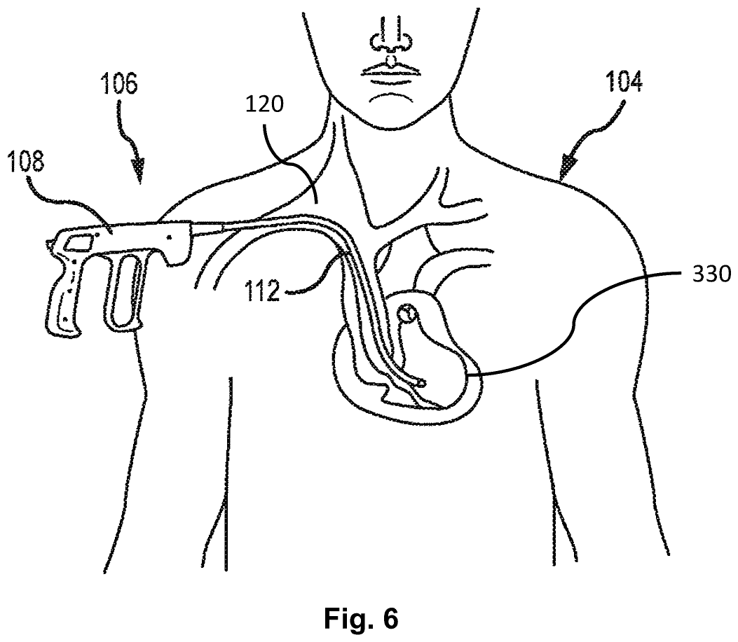

FIG. 5 is a perspective side view of the distal end of an example sheath assembly 112, according to aspects of the present disclosure. In this example, the blade 1012 of the cutting tip 632 is extended beyond the distal end of the outer band 636. Also visible is the flexible outer jacket 628. In this configuration, the blade 1012 can be advanced against tissue in order to cut it (e.g., to separate the tissue from an implanted lead).

FIG. 6 is a diagrammatic view of a surgical device 106 that has been introduced into a body lumen 334 of a patient 104 to remove an implanted lead 330, according to aspects of the present disclosure. The lead 330 may be surrounded by or embedded in tissue, which may be separated from the lead by the surgical device 106 as described herein. The lead may then be drawn into a lumen of the surgical device (e.g., lumen 300 of FIG. 3) for removal from the body lumen 334.

FIG. 7 is a side cross-sectional view of an example sheath assembly 112 removing a lead 330 from a body lumen 334, according to aspects of the present disclosure. After being implanted in the body lumen 334 for a period of time, the lead 330 may be partially or completely surrounded by tissue 338 (e.g., scar tissue) that has grown over the lead 330 within the body lumen 334. The tissue 338 may be attached or adhered to both the lead 330 and the wall of the body lumen 334, thus making the lead 330 difficult to safely remove from the body lumen 334.

In order to remove the lead safely, a clinician may advance the sheath assembly 112 over a portion the lead 330 such that the lead 330 at least partially enters the lumen 300 of the sheath assembly 112. The sheath assembly may then be further advanced until it contacts the tissue 338, at which point the outer band 636 may be used to dilate the tissue, and/or the cutting tip 632 may be extended distal of the outer band such that the blade of the cutting tip (e.g., blade 1012 of FIG. 5) rotates and/or translates in contact with the tissue 338, thus cutting the tissue. Through a combination of dilation and cutting, the sheath assembly 112 may thus form a gap 700 between the tissue 338 and the lead 330. In cases where the tissue 338 completely surrounds the lead 330, the gap 700 may for example be a circular or cylindrical gap that is roughly concentric with the sheath assembly 112. When the gap 700 had been advanced past either an end of the lead 330 or an end of the tissue overgrowth 338, the lead may no longer be adhered, and may be safely removable from the body lumen 334.

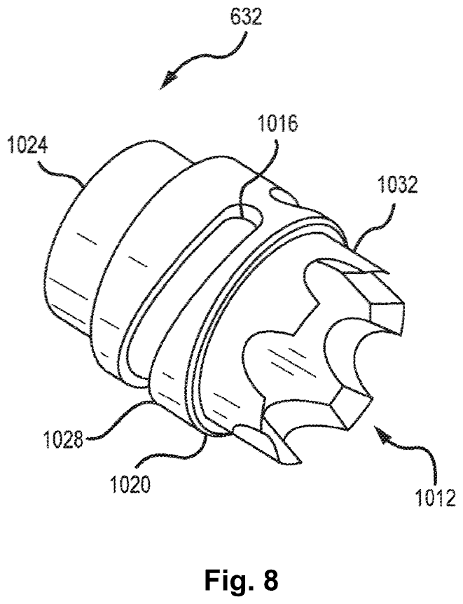

FIG. 8 is a side front perspective view of an example cutting tip 632, according to aspects of the present disclosure. The cutting tip 632 has a generally hollow cylindrical shape. The cutting tip 632 comprises a proximal portion 1024, an intermediate portion 1028, and a distal portion 1032. The outside diameter of the proximal portion 1024 is sized to allow it to be inserted to and/or engage (or otherwise attached to) the interior diameter of the flexible inner sheath (e.g., flexible inner sheath 620 of FIG. 3). The distal end of cutting tip 632 comprises a blade or cutting surface 1012, which may for example have a serrated, sharp blade profile. The intermediate portion 1028 comprises a channel or cam slot 1016 cut within its exterior surface.

As the inner flexible sheath rotates and translates within the outer sheath (e.g., flexible outer sheath 624 of FIG. 3), the outer sheath and pin may remain stationary. If so, the inner sheath, which is connected to cutting tip 632, forces the cutting tip 632 to rotate. The cam slot 1016 engages the guide pin, and the shape and profile of the cam slot 1016 controls the rate and distance with which the cutting tip 632 travels longitudinally. That is, the configuration of the cam slot 1016 controls the cutting tip's direction and amount of longitudinal travel as the cutting tip 632 is rotated, such as moving distally toward an extended position and/or proximally toward a retracted position, while the cutting tip 632 rotates in either a clockwise or counterclockwise direction.

In some aspects, the cutting tip 632 may also comprise a step up 1020 such that the outer diameter of the intermediate portion 1028 is greater than the outer diameter of the distal portion 1032, thus preventing the intermediate portion 1028 from fitting within the inner diameter of the inner sheath. As the cutting tip 632 rotates, and the blade or cutting surface 1012 extends beyond the distal end of the outer band into an extended position, the step up 1020 of the cutting tip 632 contacts the abutment of the outer band, thereby limiting the distance that the cutting tip 632 may travel and/or preventing the cutting tip 632 from exiting or extending beyond the distal tip of the outer sheath assembly, particularly the outer band, in the event that the guide pin is sheared.

The profile of the cam slot in the cutting tip may have various configurations, such as those disclosed in U.S. patent application Ser. No. 13/834,405 filed Mar. 15, 2013 and entitled Retractable Blade For Lead Removal Device, which is hereby incorporated herein by reference in its entirety as though fully set forth herein. For example, the cam slot 1016 may have a substantially linear profile, a substantially sinusoidal profile, or a combination of linear and non-linear profiles. Additionally, the cam slot 1016 may have an open and continuous configuration, thereby allowing the cutting tip to continuously rotate, or the cam slot may have a closed and discontinuous configuration such that when the cutting tip reaches its fully extended position, the trigger of the handle assembly may be released or reversed so that the cutting tip returns to initially retracted position before being re-actuated. For instance, the cam slot 1016 in FIG. 8 is discontinuous because the cam slot does not travel around the entire circumference of the exterior of the cutting tip 632.

Although certain figures in this disclosure only illustrate either the open or closed cam slot configuration, either configuration may be used with any of the aspects disclosed and/or discussed herein and are considered within the scope of this disclosure. Furthermore, various types of cam slots 1016, such as a partial lobe cam (which includes a cam slot 1016 surrounding less than 360 degrees of the circumference of the exterior surface of the cutting tip 632), a single lobe cam (which includes a cam slot 1016 surrounding 360 degrees of the circumference of the exterior surface of the cutting tip 632), a double lobe cam (which includes a cam slot 1016 surrounding 720 degrees of the circumference of the exterior surface of the cutting tip 632) and/or other multiple lobe cams.

The distal end of cutting tip 632 may comprise a cutting surface 1012 having different blade profiles, such as those disclosed in U.S. patent application Ser. No. 13/834,405 filed Mar. 15, 2013 and entitled “Retractable Blade For Lead Removal Device”, which is hereby incorporated herein by reference in its entirety as though fully set forth herein. For example, the plane of the blade or cutting surface 1012 of the distal end 1032 of the cutting tip 632 depicted in the figures of this disclosure is parallel to the plane of the proximal end 1024 of the cutting tip 632. The plane of the cutting surface, however, may be offset (0 degrees to 90 degrees) from the plane of the proximal end 1024 of the cutting tip 623. Also, as discussed above, the profile of the cutting surface 1012 shown in FIG. 8 includes a plurality of serrations. However, depending on the implementation, the profile of the cutting surface 1012 need not be serrated, and may comprise other configurations, such as a constant and/or smooth sharp profile. The profile of the cutting surface 1012 in FIG. 8 includes 6 serrations. However, it may be desirable to have other numbers of serrations, such as 4, 5, 7, 8, 10, or more serrations. Furthermore, the serrations may comprise a myriad of different shapes and configurations, including but not limited to any variation of a square, rectangle, rhombus, parallelogram, trapezoid, triangle, circle, ellipse, kite, etc.

As discussed above, FIG. 8 depicts the intermediate portion 1028 of the cutting tip 632 having a cam slot (or channel) 1016 cut within its exterior surface, as a means of controlling longitudinal motion of the cutting tip 632 as the cutting tip 632 is rotated. It should be understood that this mechanism is presented here for exemplary purposes, and that other means of rotating and/or translating the cutting tip 632 may be used instead or in addition, without departing from the spirit of the present disclosure, so long as at least some of the methods described herein can be performed.

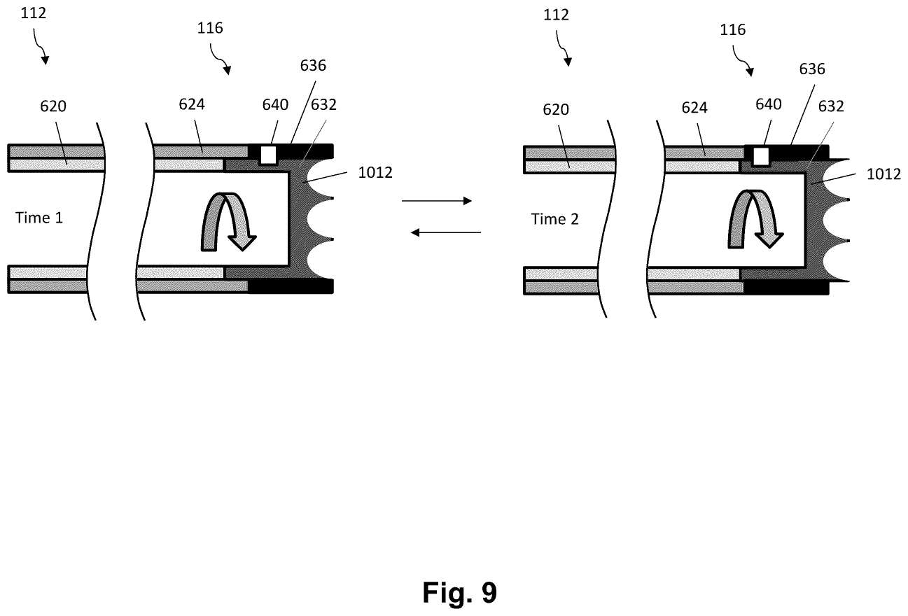

FIG. 9 is a schematic cross-sectional view of the reciprocating motion of a cutting tip 632 of the distal portion 116 of an example sheath assembly 112, according to aspects of the present disclosure. Visible is the guide pin 640, which is fixedly attached to the outer band 636, which is fixedly attached to a distal end of the flexible outer sheath 624. The flexible outer sheath 624 surrounds the flexible inner sheath 620, to whose distal end the cutting tip 632 is fixedly attached. Due to the motion of the guide pin 640 in the cam slot 1016 as the flexible inner sheath and cutting tip 632 (as described above), the cutting tip 632 may move longitudinally as it rotates, such that at a first time (“Time 1”) the blade 1012 of the cutting tip 632 is in a retracted position within the outer band 636, while at a second time the blade 1012 of the cutting tip 632 is in an extended or cutting position wherein the blade 1012 projects beyond a distal end of the outer band 636. Depending on the implementation, rotation of the cutting tip 632 relative to the outer band 636 may cause the blade 1012 to oscillate between the extended and retracted positions, either by continuous rotation in one direction (e.g., clockwise or counterclockwise) or by oscillating rotation in alternating directions. In some examples, the retracted position may represent a “home” position for the blade 1012, such that when the trigger of the handle assembly is released, the flexible inner sheath 620 and the cutting tip 632 are automatically rotated to a “home” clock angle wherein the cutting tip 632 is translated to a longitudinal position wherein the blade 1012 of the cutting tip 632 is behind the outer band 636 and thus protected from cutting tissues of the patient.

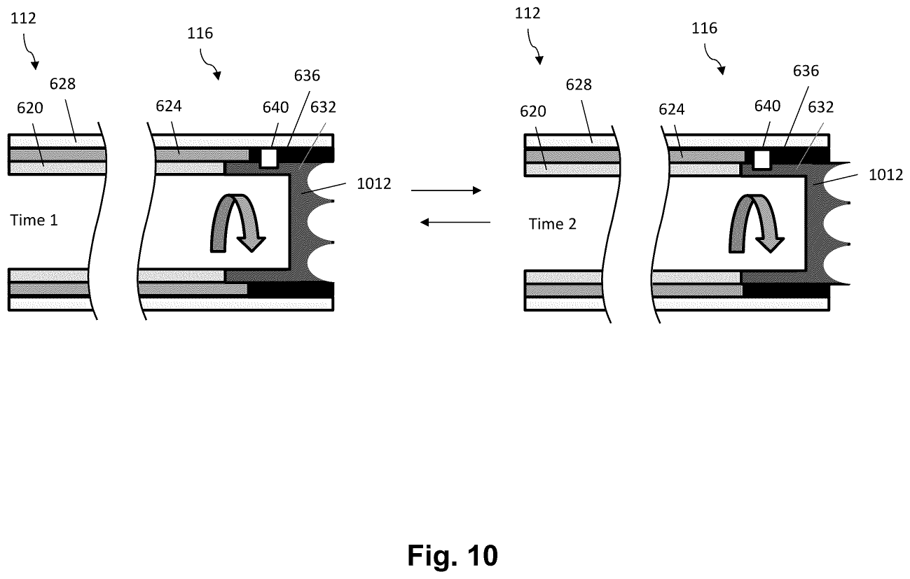

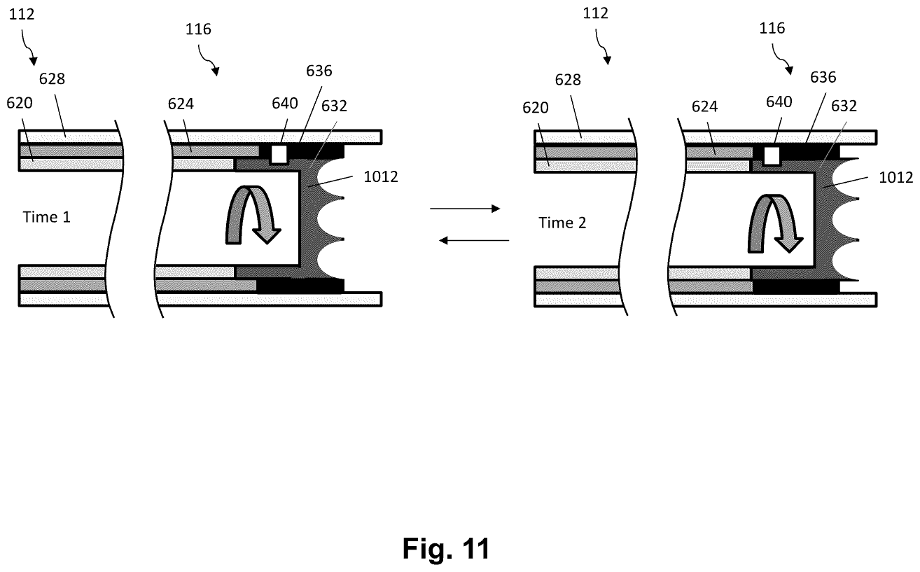

FIG. 10 is a schematic cross-sectional view of the reciprocating motion of a cutting tip 632 of the distal portion 116 of the example sheath assembly 112 of FIG. 9, according to aspects of the present disclosure. Visible are the guide pin 640, outer band 636, flexible outer sheath 624, flexible inner sheath 620, cutting tip 632, and blade 1012. Also visible in FIG. 10 is an outer jacket or guard 628, which surrounds the flexible outer sheath 624. The outer jacket or guard 628 may extend from a proximal end to a distal end of the sheath assembly 112, and may be manually extendable or retractable by the clinician, such that in its fully retracted position (shown here in FIG. 10) the outer jacket or guard 628 does not extend beyond the distal end of the outer band 636. In a fully extended position (shown below in FIG. 11), a distal end of the outer jacket or guard 628 may extend distal of the distal end of the outer band 636. In this configuration, the reciprocating action of the cutting tip 623 and blade 1012 can proceed as described above in FIG. 9.

FIG. 11 is a schematic cross-sectional view of the reciprocating motion of a cutting tip 632 of the distal portion 116 of the example sheath assembly 112 of FIG. 10, according to aspects of the present disclosure. Visible are the guide pin 640, outer band 636, flexible outer sheath 624, flexible inner sheath 620, cutting tip 632, and blade 1012. Also visible in FIG. 11 is the outer jacket or guard 628, which surrounds the flexible outer sheath 624. In the example of FIG. 11, the outer jacket or guard 628 is in an extended position, such that the distal end of the outer jacket or guard 628 extends distal of the distal end of the outer band 636, by an amount sufficient to cover the blade 1012 of the cutting tip 632, even when the blade 1012 is in its fully extended position. A clinician may for example place the outer jacket or guard 628 in this position such that if an accidental trigger press occurs, resulting in rotation and longitudinal translation of the cutting tip 632, the blade 1012 will nevertheless be protected from cutting tissues of the patient. In other instances, the shield may permit the blade to cut tissue while shielded, reducing potential to cut the vessel wall or lead. Tissue may be pulled into the cutting mechanism in this manner.

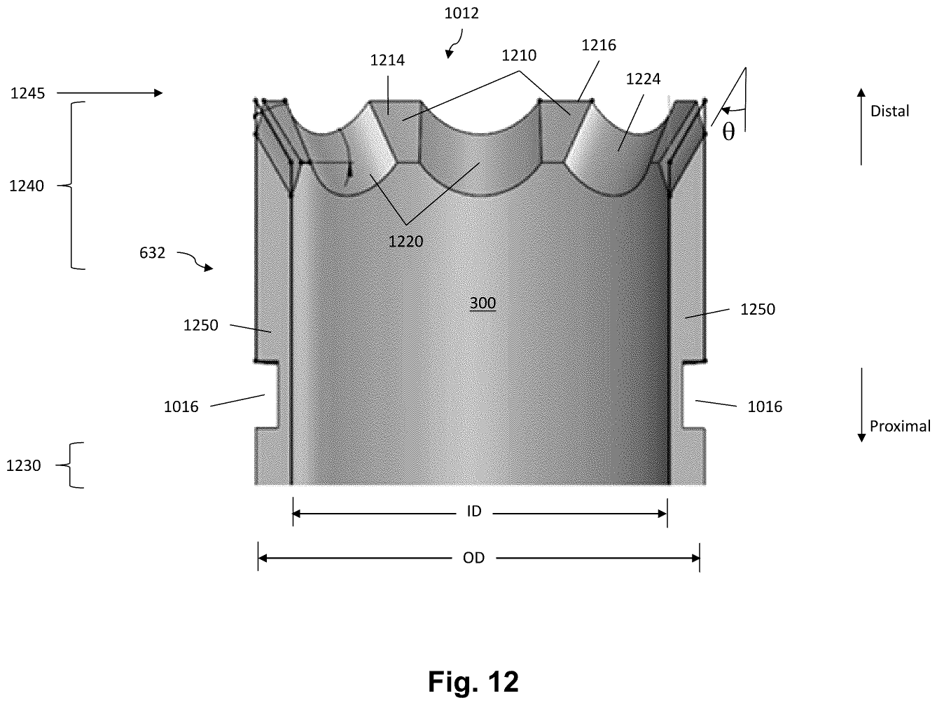

FIG. 12 is a side cross-sectional view of the cutting tip 632 of FIG. 8, according to aspects of the present disclosure. Visible are the cam slot 1016, inner lumen 300, and blade 1012. The cutting tip has an outer diameter OD and an inner diameter ID (e.g., the diameter of the inner lumen 300), as well as sidewall 1250, a proximal portion 1230, and a distal portion 1240 terminating at a distal end 1245. The blade includes a number of teeth 1210 and serrations 1220. Each tooth 1210 has a trapezoidal face 1214 chamfered inward toward the inner lumen 300, and a sharp distal edge 1216. Depending on the implementation, the length of the edges 1216 may be different than shown in FIG. 12. For example, the edges 1216 may be wider or narrower, or may form sharp points, or may be of more than one length. Each serration 1220 includes a curved surface 1224 (e.g., a hemicylindrical surface or other curved surface) chamfered inward toward the outer lumen. The chamfer of the teeth 1210 and serrations 1220 forms an angle of θ degrees with the vertical. In an example, θ is equal to −30 degrees, although other values both larger and smaller may be used instead or in addition.

The inward-facing chamfer (which may also be referred to as a negative chamfer) advantageously puts the sharp distal edges 1216 of the teeth 1210 as far as possible from the lead for a given outer diameter OD, such that as the lead passes into the inner lumen 300 of the cutting tip 632, the risk of accidentally cutting or snagging the lead is minimized.

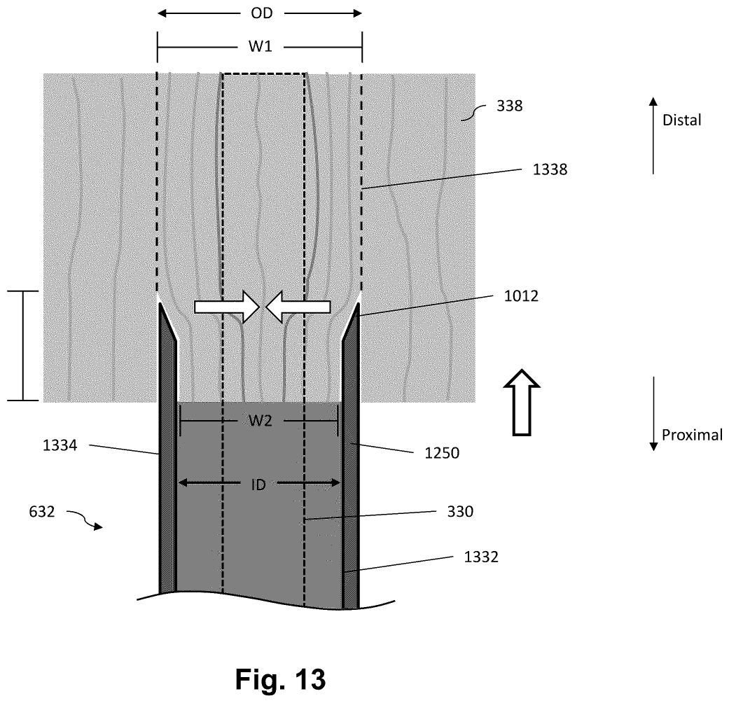

FIG. 13 is a schematic, diagrammatic, side cross-sectional view of an example cutting tip 632 cutting into tissue 338 surrounding a lead 330, according to aspects of the present disclosure. As the cutting tip 632 advances distally through the tissue 338, it cuts a cylindrical plug 1338 that includes both the lead 330 and a portion of the tissue 338 surrounding the lead 330. The tip of the blade 1012 is aligned with the outer wall surface 1334 of the cutting tip 632 (e.g., the outer surface of the wall 1250). Thus, at the tip of the blade 1212, the plug 1338 has a diameter or width W1, which is equal to the outer diameter OD of the cutting tip 632. However, because of the inward chamfer or negative chamfer of the blade 1012, as the plug 1338 moves proximally into the cutting tip 632, the plug must compress to a smaller diameter or width W2, which is equal to the inner diameter ID of the cutting tip 632. Because the lead 330 is relatively incompressible as compared with the tissue 338, the compression of the plug 1338 may be considered primarily a compression of the tissue component of the plug 1338. This compression creates an outward force between the plug 1338 and the inner wall surface 1332 of the cutting tip 632 (e.g., the inner surface of the wall 1250), thus leading to friction.

It is understood that a second shaft or outer sheath 624 and/or a third shaft or outer jacket 628, although not shown in FIG. 13, can also be provided (e.g., as shown in FIGS. 9-11).

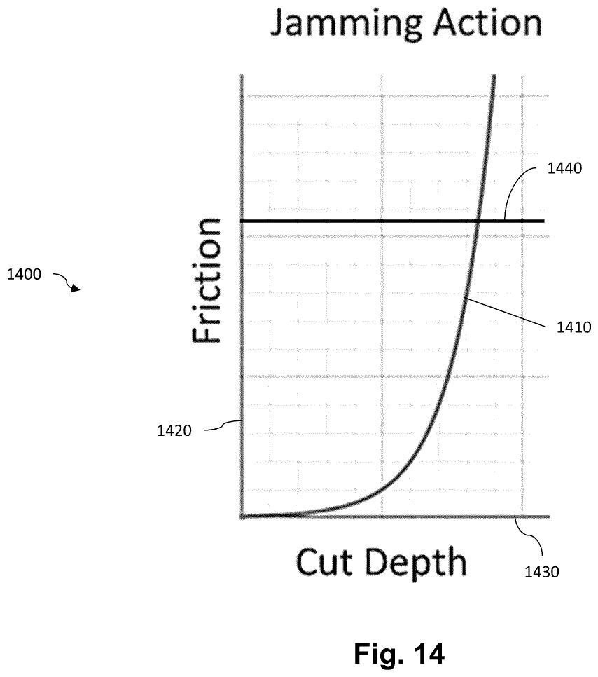

FIG. 14 is a graph 1400 showing a curve 1410 of compressive friction force 1420 as a function of the cut depth 1430 of the cutting tip into the tissue surrounding the lead, according to aspects of the present disclosure. The compressive friction force 1420 is the component of total friction that is caused by compression of the tissue inside the cutting tip. It is understood that other contributions to the total friction force may exist. As can be seen in the graph 1400, the compressive friction force 1420 increases exponentially with cut depth 1430 until it exceeds a jamming threshold 1440. Once the jamming threshold 1440 is exceeded, the cutting tip may jam within the tissue, making further distal advancement of the cutting tip into the tissue increasingly difficult, or even impossible, without risk to surrounding tissues such as the wall of the heart. Removal of the cutting catheter may also become more difficult once the cutting tip has jammed. Thus, a need exists for improved cutting tips that generate less compressive friction or no compressive friction and are thus less prone to jamming.

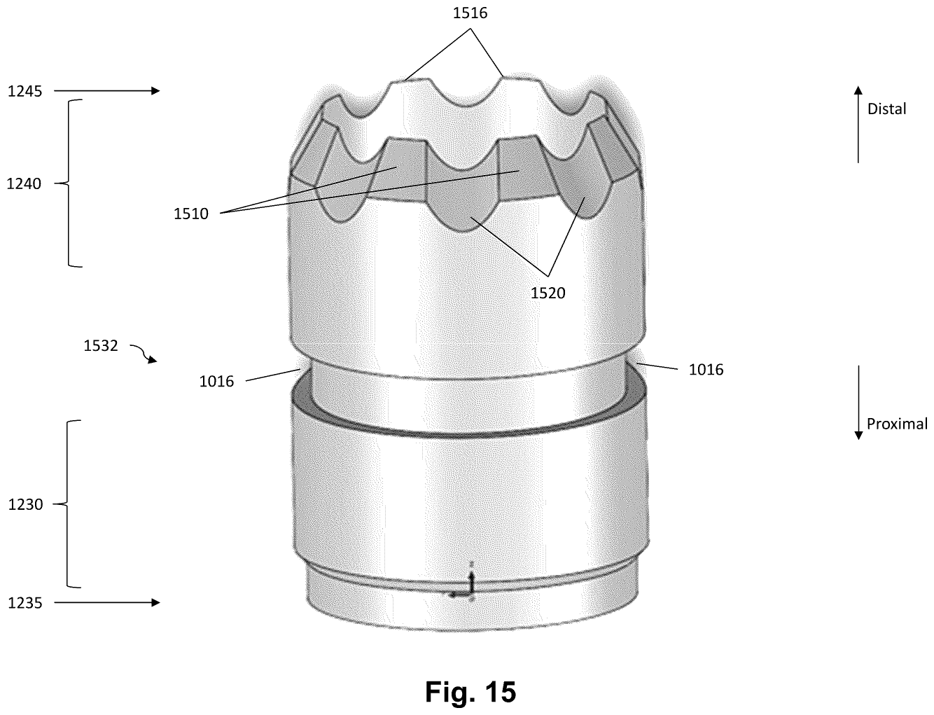

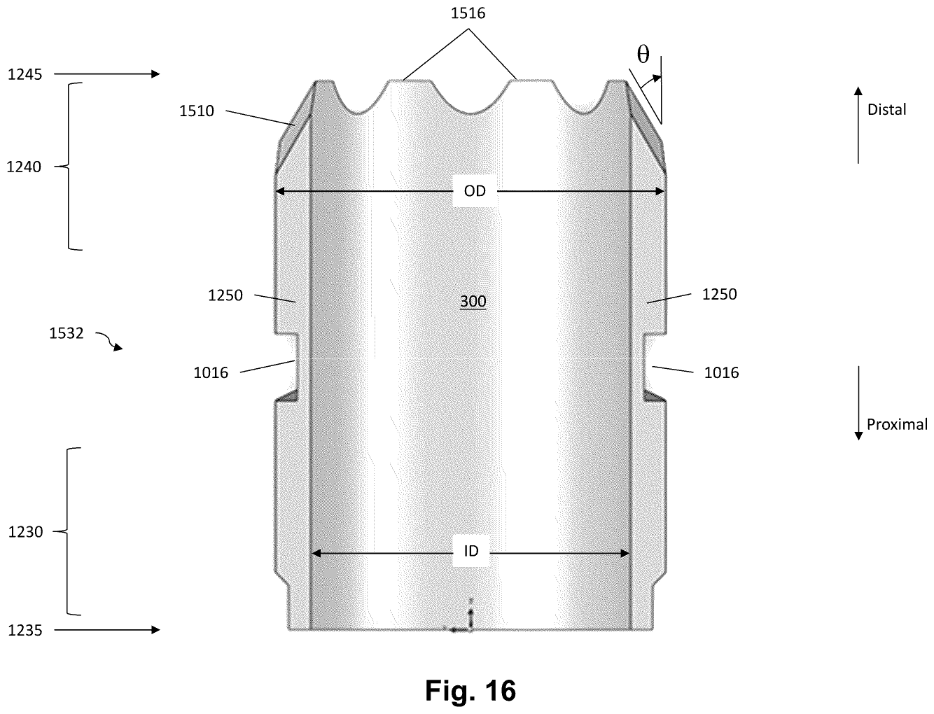

FIG. 15 is a side perspective view of an example cutting tip 1532 that incorporates an outward chamfer or positive chamfer, according to aspects of the present disclosure. As with the inward-chamfered cutting tip of FIGS. 8 and 12-13, the outward-chamfered cutting tip 1532 includes trapezoidal teeth 1510, scalloped or hemicylindrical serrations 1520, and sharp distal cutting edges 1516. However, in the example shown in FIG. 15, the cutting edges 1516 are aligned with the inner diameter rather than the outer diameter of the cutting tip, as shown below in FIG. 16, leading to less compressive friction. Also visible are the cam slot 1016, proximal portion 1230, proximal end 1235, distal portion 1240, and distal edge 1245. The proximal end 1235 of the cutting tip can be fixedly attached to and/or otherwise coupled to the distal end of the first shaft of inner sheath 620 (FIG. 1), as shown, for example, in FIGS. 9, 10, and 11. Rotation of the inner sheath 620 causes corresponding rotation of the cutting tip, as described herein.

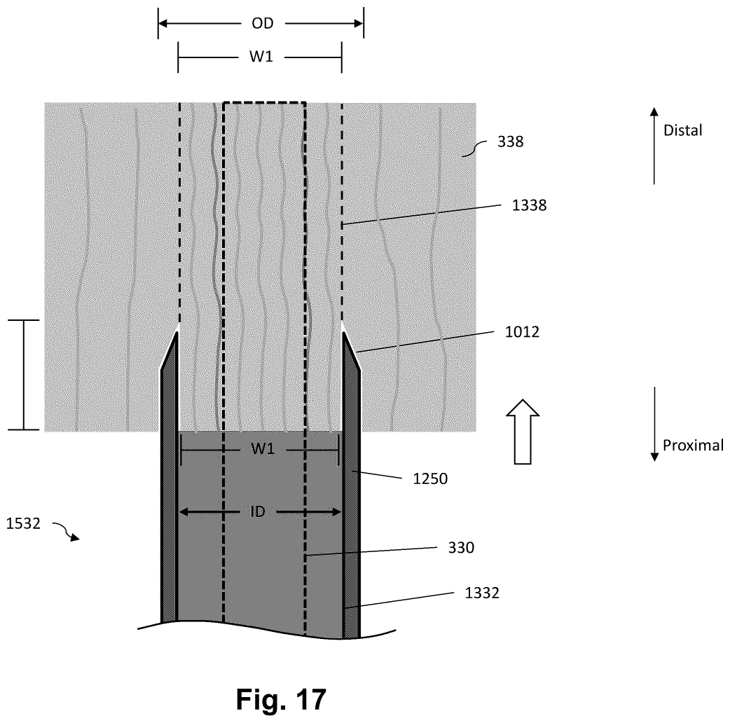

FIG. 16 is a side cross-sectional view of an example cutting tip 1532 that incorporates an outward chamfer or positive chamfer, according to aspects of the present disclosure. Visible are the sharp cutting edges 1516, outer diameter OD, inner diameter ID, and cam slot 1016. In the example shown in FIG. 16, the cutting edges 1516 are aligned with the inner diameter rather than the outer diameter of the cutting tip, thus leading to less or no compression of the tissue pulled into the inner lumen 300, as shown below in FIG. 17. Each tooth 1510 forms a chamfer angle of θ degrees with the vertical. In an example, θ is equal to +30 degrees, although other values both larger and smaller may be used instead or in addition.

Also visible are the proximal portion 1230, proximal end 1235, distal portion 1240, distal edge 1245, and sidewall 1250.

FIG. 17 is a schematic, diagrammatic, side cross-sectional view of an example outward-chamfered cutting tip 1532 cutting into tissue 338 surrounding a lead 330, according to aspects of the present disclosure. As the outward-chamfered cutting tip 1532 advances distally through the tissue 338, it cuts a cylindrical plug 1338 that includes both the lead 330 and a portion of the tissue 338 surrounding the lead 330. At the tip of the blade 1012, the plug 1338 has a diameter or width W1, which is equal to the inner diameter ID of the cutting tip 1532. Because of the outward chamfer or positive chamfer of the blade 1012, as the plug 1338 moves proximally into the cutting tip 1532, the plug moves into the interior lumen of the cutting tip, which also has an inner diameter ID, and thus the plug's width W2 inside of the cutting tip 1532 is approximately equal to its width W1 outside of the cutting tip 1532. Thus, little or no compression of the tissue 338 takes place. This lack of compression means there is little or no outward force between the plug 1338 and the inner wall surface 1332 of the cutting tip 1532 (e.g., the inner surface of the wall 1250), and thus little or no compressive friction.

In an example, the distal end of the inner sheath is coupled to the proximal end of the cutting tip, and the inner diameter of the inner sheath is equal to or greater than the inner diameter of the cutting tip, and thus there is also little or no compression at or proximal of the transition between the cutting tip and the inner sheath. It is understood that a second shaft or outer sheath 624 and/or a third shaft or outer jacket 628, although not shown in FIG. 17, can also be provided (e.g., as shown in FIGS. 9-11).

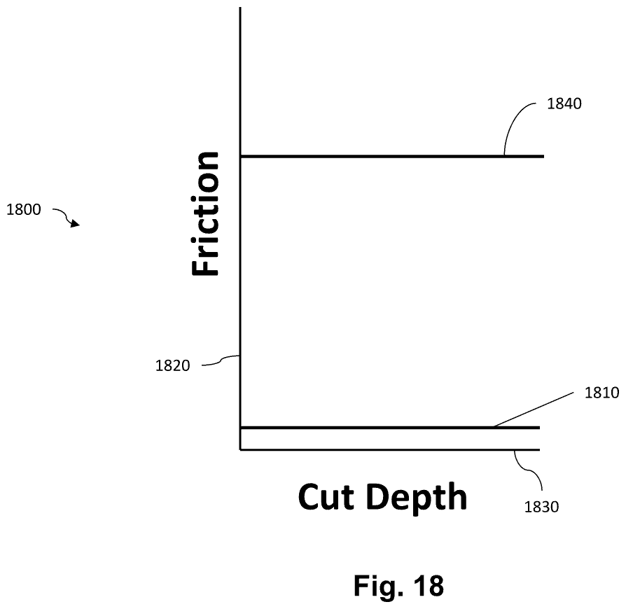

FIG. 18 is a graph 1800 showing a curve 1810 of compressive friction force 1820 as a function of the cut depth 1830 of the outward-chamfered cutting tip into the tissue surrounding the lead, according to aspects of the present disclosure. As can be seen in the graph 1800, the compressive friction force 1820 has a small or even zero value that does not substantially increase with cut depth 1830 and does not approach the jamming threshold 1840. Thus, the outward-chamfered cutting tip can be seen as substantially less likely to jam than the inward-chamfered cutting tip of FIG. 14.

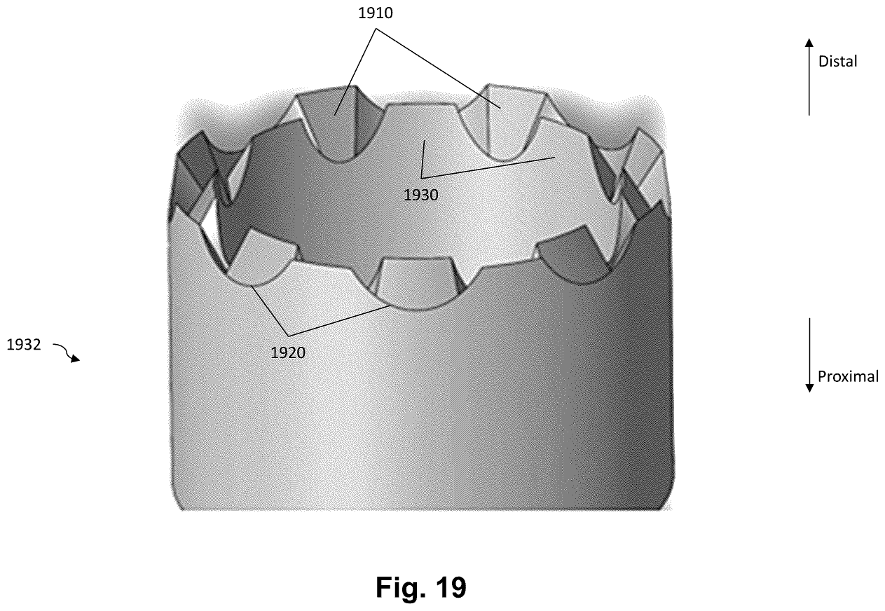

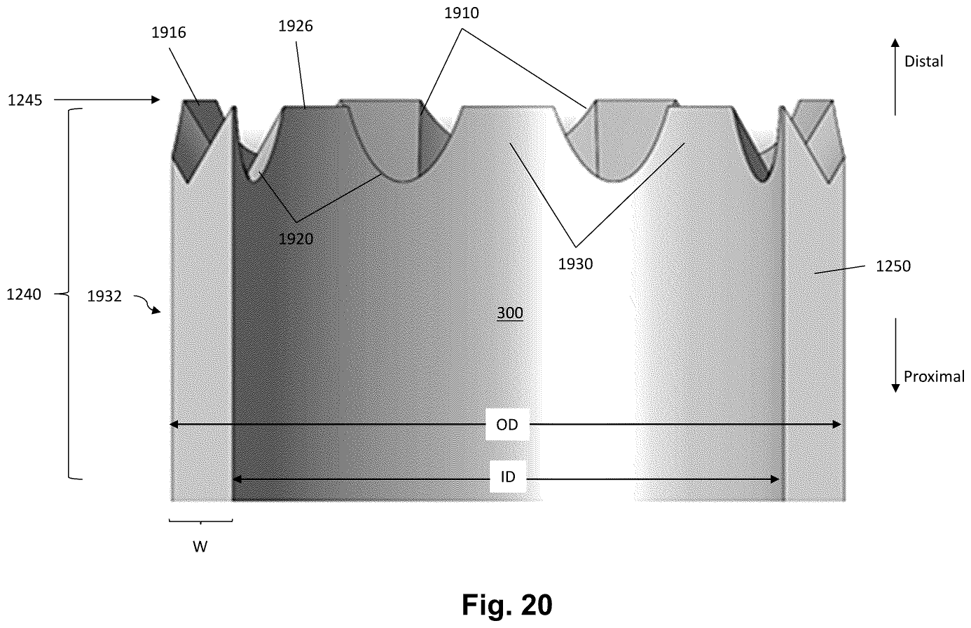

FIG. 19 is a side perspective view of an example cutting tip 1932 that incorporates a dual chamfer or positive-negative chamfer, according to aspects of the present disclosure. The dual-chamfered cutting tip includes a row of inner teeth 1930 with an outward-facing chamfer and a row of outer teeth 1910 with an inward-facing chamfer. Both the inner teeth 1930 and the outer teeth 1910 are separated by serrations 1920. In the example shown in FIG. 19, the inner teeth 1930 alternate with the outer teeth 1910, but in other aspects the inner teeth and outer teeth may be aligned, or may be circumferentially offset from one another by a different amount than that shown in FIG. 19. This dual row of cutting teeth can increase the width of the cut formed by the teeth, thus reducing or eliminating compression of the tissue as shown below in FIG. 20. In an example, the chamfer angles of the outer teeth are −30 degrees and the chamfer angles of the inner teeth are +30 degrees, although other values both larger and smaller may be used instead or in addition. It is noted that different teeth may have different chamfer angles, and the angles of the outer teeth need not be negatives of the angles of the inner teeth.

FIG. 20 is a side cross-sectional view of the dual-chamfered cutting tip 1932 of FIG. 19, according to aspects of the present disclosure. Visible are the outer teeth 1910, inner teeth 1930, serrations 1920, and inner lumen 300. In the example shown in FIG. 20, the distal cutting edges 1916 of the outer teeth 1910 are aligned with the outer diameter OD, while the distal cutting edges 1926 of the outer teeth 1910 are aligned with the inner diameter. The radial distance between the outer edges 1916 and the inner edges 1926 is therefore equal to OD-ID, or the width W of the wall 2010 of the cutting tip 1932. When tissue is cut by the cutting tip 1932, an annular region of width W surrounding the central lumen 300 is pulverized. A portion of the pulverized material may enter the bloodstream, awhile another portion may exit through the lumen 300.

Also visible are the distal portion 1240, distal edge 1245, and sidewall 1250.

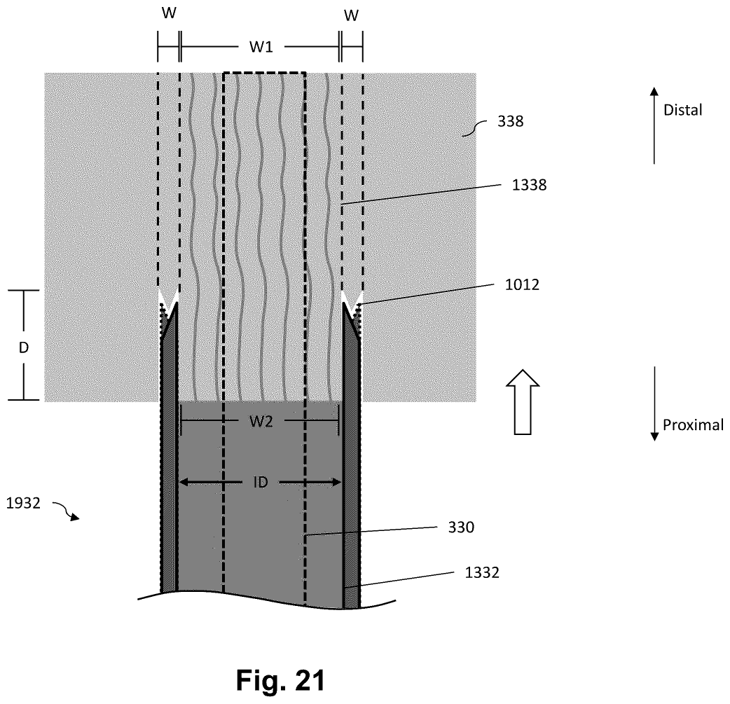

FIG. 21 is a schematic, diagrammatic, side cross-sectional view of an example dual-chamfered cutting tip 1932 cutting into tissue 338 surrounding a lead 330, according to aspects of the present disclosure. As the dual-chamfered cutting tip 1932 advances distally through the tissue 338 to a cutting depth D, it cuts a cylindrical plug 1338 that includes both the lead 330 and a portion of the tissue 338 surrounding the lead 330. An annular region of width W, surrounding the inner diameter ID, is pulverized by the dual-chamfered inner and outer blades, as described above.

At the tip of the blade 1012, the plug 1338 has a diameter or width W1, which is equal to the inner diameter ID of the cutting tip 1932. Because of the dual chamfer of the blade 1012, as the plug 1338 moves proximally into the cutting tip 1532, the plug moves into the interior lumen of the cutting tip, which also has an inner diameter ID, and thus the plug's width W2 inside of the cutting tip 1932 is approximately equal to its width W1 outside of the cutting tip 1932. Thus, little or no compression of the tissue 338 takes place. This lack of compression means there is little or no outward force between the plug 1338 and the inner wall surface 1332 of the cutting tip 1932, and thus little or no compressive friction.

In an example, the distal end of the inner sheath is coupled to the proximal end of the cutting tip, and the inner diameter of the inner sheath is equal to or greater than the inner diameter of the cutting tip, and thus there is also little or no compression at or proximal of the transition between the cutting tip and the inner sheath. It is understood that a second shaft or outer sheath 624 and/or a third shaft or outer jacket 628, although not shown in FIG. 21, can also be provided (e.g., as shown in FIGS. 9-11).

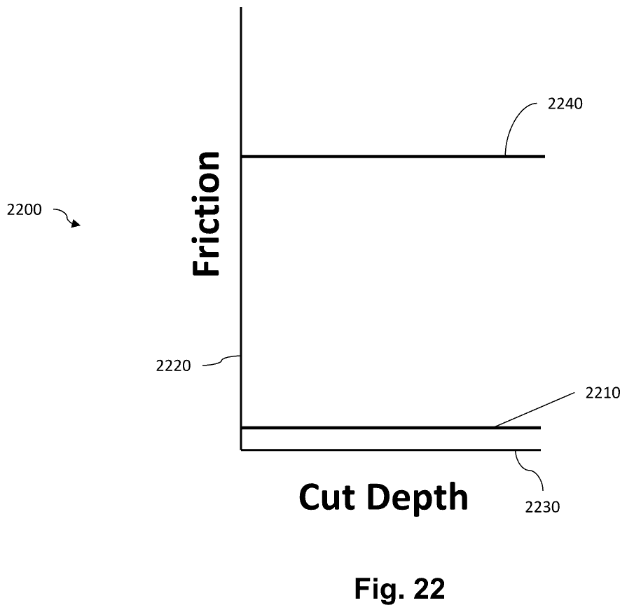

FIG. 22 is a graph 2200 showing a curve 2210 of compressive friction force 2220 as a function of the cut depth 2230 of the dual-chamfered cutting tip into the tissue surrounding the lead, according to aspects of the present disclosure. As can be seen in the graph 2200, the compressive friction force 2220 has a small or even zero value that does not substantially increase with cut depth 2230 and does not approach the jamming threshold 2240. Thus, the dual-chamfered cutting tip can be seen as substantially less likely to jam than the inward-chamfered cutting tip of FIG. 14.

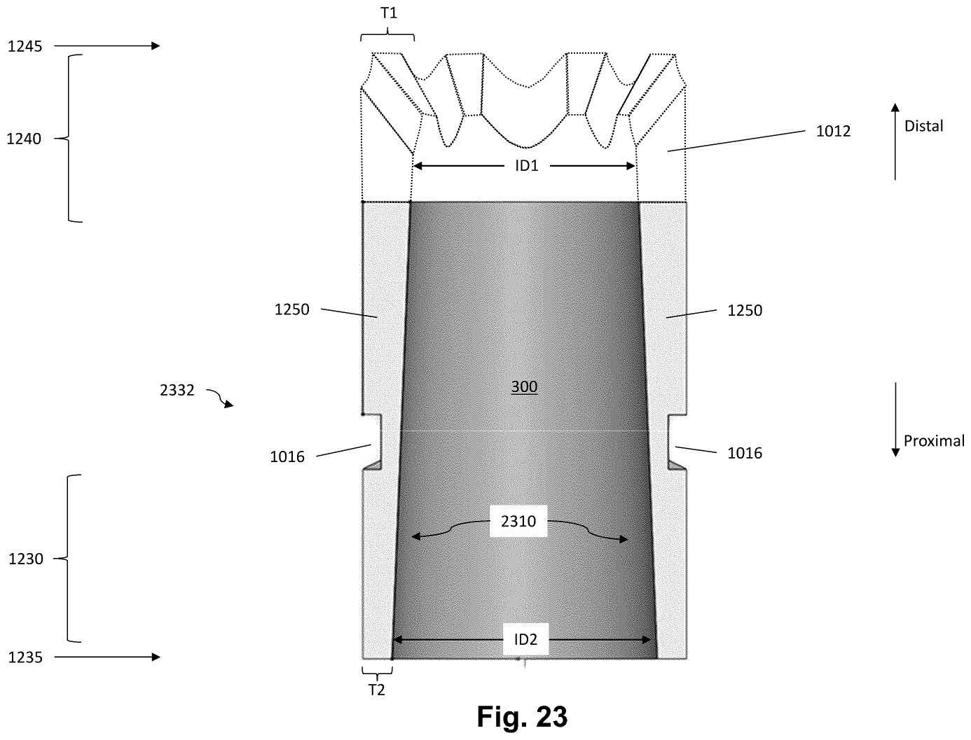

FIG. 23 is a side cross-sectional view of an example cutting tip 2332 with an inward or negative chamfer and a tapered interior lumen 300, according to aspects of the present disclosure. The blade portion 1012 of the cutting tip 2332 is similar to that of the cutting tip 632 of FIGS. 8 and 12-13. However, the interior lumen 300 of the cutting tip 2332 includes an outward taper 2310 such that the distal end of the lumen 300 has a diameter ID1, whereas the proximal end of the lumen has a larder diameter ID2. This outward taper means that as a plug of tissue moves proximally through the lumen 300, the compression of the tissue will decrease, thus limiting compression-related friction as the cutting tip 2332 is advanced distally through the tissue.

Also visible are the proximal portion 1230, proximal end 1235, distal portion 1240, distal edge 1245, and sidewall 1250. Although the cutting tip 2332 is shown with an inward or negative chamfer, it is understood that the tapered lumen of the cutting tip 2332 could also be used in conjunction with an outward or positive chamfer, or a dual chamfer, or other blade design not shown herein. Such aspects explicitly fall within the scope of the present disclosure.

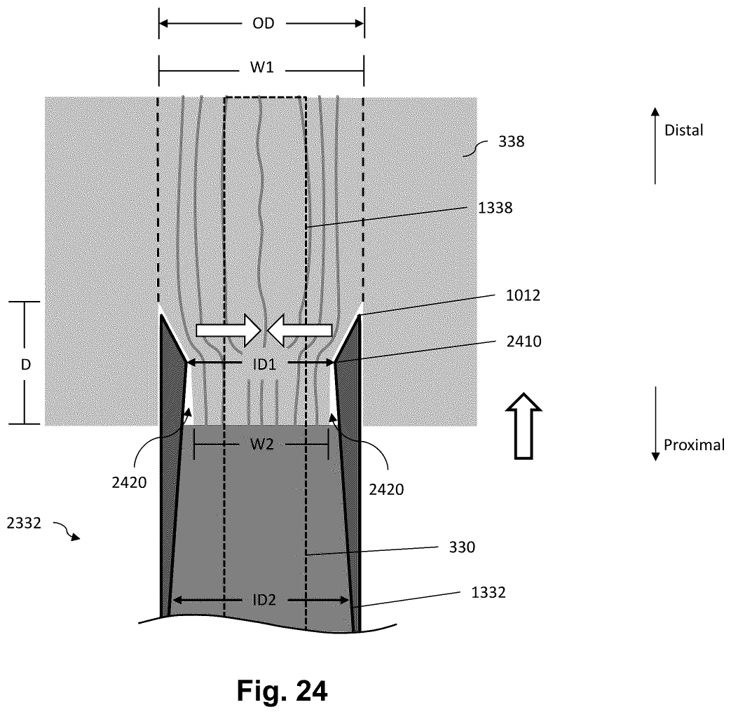

FIG. 24 is a schematic, diagrammatic, side cross-sectional view of an example inward-chamfered cutting tip 1532 with tapered lumen 300 cutting into tissue 338 surrounding a lead 330, according to aspects of the present disclosure. As the inward-chamfered cutting tip 1532 advances distally through the tissue 338, it cuts a cylindrical plug 1338 that includes both the lead 330 and a portion of the tissue 338 surrounding the lead 330. At the tip of the blade 1012, the plug 1338 has a diameter or width W1, which is equal to the outer diameter OD of the cutting tip 1532. Because of the inward chamfer or negative chamfer of the blade 1012, as the plug 1338 moves proximally into the cutting tip 1532, the plug moves into the interior lumen of the cutting tip, whose proximal end has an inner diameter ID1, and thus the plug's width W2 inside of the cutting tip 1532 is compressed until it is approximately equal to ID1. However, proximal of the lumen's distal end, the diameter of the lumen widens out until it reaches a diameter of ID2 at the proximal end of the cutting tip 2332. Thus, the distal end of the lumen forms a local/relative minimum lumen diameter 2410 where compression of the tissue is at a maximum. As tissue moves proximal of the minimum lumen diameter 2410, the compression (and thus the compression-related friction) reduces, eventually forming gaps 2420 between the plug 1338 and the wall 1332 of the cutting tip 2332. Thus, as the plug 1338 advances into the cutting tip 2332, compression and compressive friction increase until the proximal end of the plug 1338 reaches the minimum lumen diameter 2410, and remains relatively constant thereafter as the plug advances further into the cutting tip 2332.

In an example, the distal end of the inner sheath is coupled to the proximal end of the cutting tip, and the inner diameter of the inner sheath is equal to or greater than the inner diameter of the cutting tip, and thus there is also little or no compression at or proximal of the transition between the cutting tip and the inner sheath. It is understood that a second shaft or outer sheath 624 and/or a third shaft or outer jacket 628, although not shown in FIG. 24, can also be provided (e.g., as shown in FIGS. 9-11).

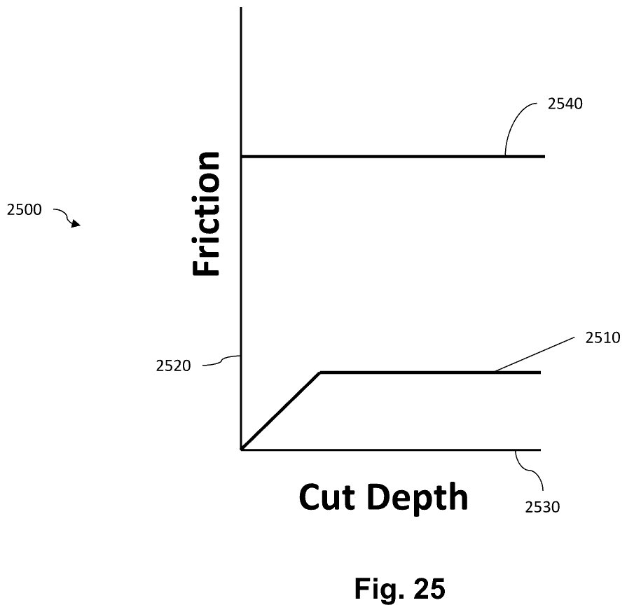

FIG. 25 is a graph 2500 showing a curve 2510 of compressive friction force 2520 as a function of the cut depth 2530 of the dual-chamfered cutting tip into the tissue surrounding the lead, according to aspects of the present disclosure. As can be seen in the graph 2500, the compressive friction force 2220 initially has a small or zero value that increases with cut depth 2330 until the plug has reached the pinch point, and does not substantially increase with cut depth 2230 thereafter, and thus does not approach the jamming threshold 2540. Thus, the inward-chamfered, tapered-lumen cutting tip can be seen as substantially less likely to jam than the inward-chamfered, straight-lumen cutting tip of FIG. 14.

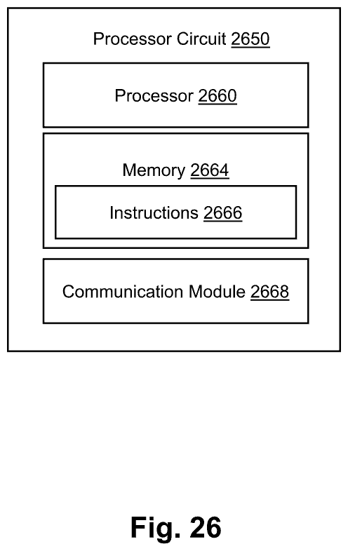

FIG. 26 is a schematic diagram of a processor circuit 2650, according to aspects of the present disclosure. The processor circuit 2650 may be implemented in the intraluminal cutting device 106, or other devices or workstations (e.g., third-party workstations, network routers, etc.), or in a cloud processor or other remote processing unit, as necessary to implement the method. As shown, the processor circuit 2650 may include a processor 2660, a memory 2664, and a communication module 2668. These elements may be in direct or indirect communication with each other, for example via one or more buses.

The processor 2660 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 2660 may also comprise another hardware device, a firmware device, or any combination thereof configured to perform the operations described herein. The processor 2660 may also be implemented as a combination of computing devices, e.g., a combination of a DSP and a microprocessor, a plurality of microprocessors, one or more microprocessors in conjunction with a DSP core, or any other such configuration.

The memory 2664 may include a cache memory (e.g., a cache memory of the processor 2660), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 2664 includes a non-transitory computer-readable medium. The memory 2664 may store instructions 2666. The instructions 2666 may include instructions that, when executed by the processor 2660, cause the processor 2660 to perform the operations described herein. Instructions 2666 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 2668 can include any electronic circuitry and/or logic circuitry to facilitate direct or indirect communication of data between the processor circuit 2650, and other processors or devices. In that regard, the communication module 2668 can be an input/output (I/O) device. In some instances, the communication module 2668 facilitates direct or indirect communication between various elements of the processor circuit 2650 and/or the intraluminal cutting device 106. The communication module 2668 may communicate within the processor circuit 2650 through numerous methods or protocols. Serial communication protocols may include but are not limited to US SPI, I2C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to software updates, firmware updates, preset sharing between the processor and central server, or readings from the surgical device) may be accomplished using any suitable wireless or wired communication technology, such as a cable interface such as a USB, micro USB, Lightning, or Fire Wire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media 610 such as a USB flash drive or memory stick.

A number of variations are possible on the examples and aspects described above. For example, the technology described herein may be applied to cutting catheters and cutting tip blades of diverse types, whether currently in existence or hereinafter developed. All aspects described herein could be used in existing or novel medical procedures, including but not limited to lead extraction procedures. The principles described herein could be utilized in almost any industry that uses a round rotating cutter, including but not limited to core sampling drills. The cutting tip designs described herein can be detected by visual inspection.

Accordingly, the logical operations making up the aspects of the technology described herein are referred to variously as operations, steps, objects, elements, components, or modules. Furthermore, it should be understood that these may occur or be performed or arranged in any order, unless explicitly claimed otherwise or a specific order is inherently necessitated by the claim language. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intraluminal cutting device. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

The above specification, examples and data provide a complete description of the structure and use of exemplary aspects of the intraluminal cutting device as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter. Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. A lead removal device, comprising:
a flexible elongate member configured to be positioned within a blood vessel of a patient, wherein the flexible elongate member comprises a proximal portion and a distal portion;
a cutting tip positioned at the distal portion of the flexible elongate member and configured to rotate, wherein the cutting tip comprises:
a blade configured to cut tissue associated with the blood vessel for removal of an electrical lead implanted in the tissue from a body of a patient;
an outer wall surface defining an outer diameter;
an inner wall surface defining a lumen with an inner diameter, wherein the lumen is configured to receive the electrical lead and the tissue cut by the blade; and
an outward chamfer such that:
a cutting edge of the blade is aligned with the inner wall surface; and
the inner diameter matches a diameter of the tissue cut by the blade and entering the lumen.

2. The intraluminal cutting device of claim 1,
wherein the blade comprises a plurality of teeth and a plurality of serrations,
wherein a distal end of each tooth comprises a cutting edge.

3. The intraluminal cutting device of claim 2,
wherein each tooth of the plurality of teeth comprises a chamfered trapezoidal surface,
wherein each serration of the plurality of serrations comprises a chamfered curved surface.

4. The intraluminal cutting device of claim 1, further comprising:
a handle coupled to the proximal portion of the flexible elongate member; and,
a trigger coupled to the handle and configured such that actuation of the trigger causes the cutting tip to rotate.

5. A lead removal device, comprising:
a flexible elongate member configured to be positioned within a blood vessel of a patient, wherein the flexible elongate member comprises a proximal portion and a distal portion;
a cutting tip positioned at the distal portion of the flexible elongate member and configured to rotate, wherein the cutting tip comprises:
a blade configured to cut tissue associated with the blood vessel for removal of an electrical lead implanted in the tissue from a body of a patient;
an outer wall surface defining an outer diameter;
an inner wall surface defining a lumen with an inner diameter, wherein the lumen is configured to receive the electrical lead and the tissue cut by the blade; and
an outward chamfer and an inner chamfer such that:
a first cutting edge of the blade is aligned with the inner wall surface;
a second cutting edge of the rotating blade is aligned with the outer wall surface; and
the inner diameter matches a diameter of the tissue cut by the blade and entering the lumen.

6. The intraluminal cutting device of claim 5,
wherein the blade comprises a plurality of teeth and a plurality of serrations,
wherein a distal end of each tooth comprises a cutting edge.

7. The intraluminal cutting device of claim 6,
wherein each tooth of the plurality of teeth comprises a chamfered trapezoidal surface,
wherein each serration of the plurality of serrations comprises a chamfered curved surface.

8. The intraluminal cutting device of claim 5, further comprising:
a handle coupled to the proximal portion of the flexible elongate member; and,
a trigger coupled to the handle and configured such that actuation of the trigger causes the cutting tip to rotate.

9. An intraluminal cutting device, comprising:
a flexible elongate member configured to be positioned within a blood vessel of a patient, wherein the flexible elongate member comprises a proximal portion and a distal portion;
a cutting tip positioned at the distal portion of the flexible elongate member and configured to rotate, wherein the cutting tip comprises:
a blade configured to cut tissue associated with the blood vessel for removal of an electrical lead implanted in the tissue from a body of a patient;
an outer wall surface defining an outer diameter;
an inner wall surface defining a lumen with an inner diameter, wherein the lumen is configured to receive the electrical lead and the tissue cut by the blade, and wherein the inner wall surface comprises a taper such that:
the inner diameter at a distal end of the lumen is less than the inner diameter at a proximal end of the lumen;
the inner diameter at the proximal end of the lumen is greater than a diameter of the tissue cut by the blade and entering the lumen.

10. The intraluminal cutting device of claim 9,
wherein the blade comprises a plurality of teeth and a plurality of serrations,
wherein a distal end of each tooth comprises a cutting edge.

11. The intraluminal cutting device of claim 10,
wherein each tooth of the plurality of teeth comprises a chamfered trapezoidal surface,
wherein each serration of the plurality of serrations comprises a chamfered curved surface.

12. The intraluminal cutting device of claim 9, further comprising:
a handle coupled to the proximal portion of the flexible elongate member; and,
a trigger coupled to the handle and configured such that actuation of the trigger causes the cutting tip to rotate.

13. The intraluminal cutting device of claim 9, wherein the blade is configured such that a cutting edge of the rotating blade is aligned with the outer wall surface.

14. An intraluminal cutting device, comprising:
a flexible elongate member configured to be positioned within a body lumen of a patient, wherein the flexible elongate member comprises a proximal portion and a distal portion;
a cutting tip positioned at the distal portion of the flexible elongate member and configured to rotate, wherein the cutting tip comprises:
a blade configured to cut tissue associated with the body lumen,
an outer wall surface defining an outer diameter;
an inner wall surface defining a lumen with an inner diameter; and
wherein the cutting tip comprises a structural arrangement configured to minimize compression of the tissue cut by the blade and entering the lumen, wherein the minimization of the compression comprises at least one of minimization of compression-related friction of the blade or prevention of jamming of the blade, and
wherein the structural arrangement comprises at least one of:
the cutting tip comprises an outward chamfer or both the outward chamfer and an inward chamfer, such that the inner diameter matches a diameter of the tissue cut by the blade and entering the lumen; or
the inner wall surface comprises a taper such that:
the inner diameter at a distal end of the lumen is less than the inner diameter at a proximal end of the lumen; and
the inner diameter at the proximal end of the lumen is greater than a diameter of the tissue cut by the blade and entering the lumen.

15. The intraluminal cutting device of claim 14,
wherein the blade comprises a plurality of teeth and a plurality of serrations,
wherein a distal end of each tooth comprises a cutting edge.

16. The intraluminal cutting device of claim 15,
wherein each tooth of the plurality of teeth comprises a chamfered trapezoidal surface,
wherein each serration of the plurality of serrations comprises a chamfered curved surface.

17. The intraluminal cutting device of claim 14, further comprising:
a handle coupled to the proximal portion of the flexible elongate member, and,
a trigger coupled to the handle and configured such that actuation of the trigger canses the cutting tip to rotate.
